(19) United States
(12) Patent Application Publication
(10) Pub. No.: US 2017/0333710 A1
(43) Pub. Date: Nov. 23, 2017

(54) External Ear Nerve Stimulation

(71) Applicants: Jin Shimada, White Bear Lake, MN (US); Harry A. Puryear, Shoreview, MN (US)

(72) Inventors: Jin Shimada, White Bear Lake, MN (US); Harry A. Puryear, Shoreview, MN (US)

(21) Appl. No.: 15/493,983

(22) Filed: Apr. 21, 2017

Related U.S. Application Data
(60) Provisional application No. 62/339,330, filed on May 20, 2016.

(51) Int. Cl. A61N 1/36; A61N 1/08; A61N 1/02

(52) U.S. Cl. CPC A61N 1/36025; A61N 1/36021; A61N 1/025; A61N 1/08

(57) ABSTRACT

An electrical ear nerve stimulation earpiece apparatus includes an earpiece body configured to fit on or in the ear. A first electrode is coupled to a surface of the earpiece configured to stimulate a cranial nerve, and a second electrode is coupled to a surface of the earpiece configured to stimulate a spinal nerve. Circuitry coupled to or in the earpiece is operable to provide an electrical signal to at least the first and second electrodes to treat a disorder affecting the nervous system by stimulating the spinal nerve and the cranial nerve.

### FIELD

This application claims the benefit of U.S. Provisional Application No. 62/339,330 filed May 20, 2016, the contents of which are herein incorporated by reference.

The invention relates generally to neural stimulation of the ear, and more specifically to externally stimulating ear nerves to address nervous system and brain disorders.

BACKGROUND

A variety of medical conditions, including migraine headaches, depression, Parkinson's disease, post-traumatic stress disorder, and the like are believed or known to be related to abnormal activity in the central nervous system and the brain. Although the mechanisms of each of these disorders vary, they often involve multiple mechanisms within the central nervous system, resulting in symptoms such as pain, numbness, or loss of motor control.

The human body's nervous system includes both the somatic nervous system that provides sense of the environment (vision, skin sensation, etc.) and regulation of the skeletal muscles, and is largely under voluntary control, and the autonomic nervous system, which serves mainly to regulate the activity of the internal organs and adapt them to the body's current needs, and which is largely not under voluntary control. The autonomic nervous system involves both afferent or sensory nerve fibers that can mechanically and chemically sense the state of an organ, and efferent fibers that convey the central nervous system's response (sometimes called a reflex arc) to the sensed state information. In some cases, the somatic nervous system is also influenced, such as to cause vomiting or coughing in response to a sensed condition or as a symptom of a neurological disorder.

Treatment of neurological disorder such as migraine headaches therefore often addresses the functioning of the autonomic nervous system, such as to pharmaceutically alter or hinder certain functions or to stimulate the nervous system to reduce occurrence or alleviate symptoms of the disorder. Stimulation of the autonomic nervous system has been shown to help regulate organ function and to address other involuntary nervous system disorders and symptoms, and can also affect operation of the somatic nervous system such as reducing a sensations of pain or numbness. Because proper operation and treatment of the nervous system is an important part of managing neurological disorders such as these, it is desired to be able to provide for effective and minimally invasive stimulation of the nervous system.

### SUMMARY

In one example, an electrical ear nerve stimulation earpiece apparatus includes an earpiece body configured to fit on or in the ear. A first electrode is coupled to a surface of the earpiece configured to stimulate a cranial nerve, and a second electrode is coupled to a surface of the earpiece configured to stimulate a spinal nerve. Circuitry coupled to or in the earpiece is operable to provide an electrical signal to at least the first and second electrodes to treat a disorder affecting the nervous system by stimulating the spinal nerve and the cranial nerve.

In a further example, treating the disorder comprises modulating or suppressing signals in a user's brain that contribute to migraine headaches. The electrical signal in various examples stimulates one or more of cranial nerve 5, cervical nerves C2 and C3, and cranial nerves 7, 9, and 10. In another example, the disorder comprises migraine headaches, Parkinson's disease, epilepsy, or another neurological disorder.

In another example, the earpiece further comprises an acoustic passage configured to allow a user to hear while wearing the earpiece.

The details of one or more examples of the invention are set forth in the accompanying drawings and the description below. Other features and advantages will be apparent from the description and drawings, and from the claims.

### BRIEF DESCRIPTION OF THE FIGURES

FIG. 1 shows an external view of an ear generally at 100, along with shaded circles indicating nerve access points on and around the ear.

FIG. 2 shows a side view of an ear, including both external portions and the external ear canal comprising the portion of the ear canal before the eardrum.

FIG. 3 shows an insertable electrode earplug that can be inserted into ear canal of FIG. 2 to provide access to nerve points in the ear canal and on the external ear.

FIG. 4 shows an earpiece having electrodes to stimulate nerves in and around the ear, including an external portion that may provide power, user controls, external electrodes, grounding or signal return, and other functions.

FIG. 5 shows an earpiece coupled to a controller via an electrical cable.

### DESCRIPTION

Migraine headaches affect approximately 9% of the population of the United States, with an estimated 15% of people suffering from the condition worldwide. Although a number of forms of migraine headache have been described, all forms tend to reoccur if left untreated, and include symptoms of moderate to severe pain. Migraine headaches can be triggered by stimulations such as sound, light, smell, or other stimuli, and are often accompanied by nausea, vomiting, and increased sensitivity to external stimulation.

Most migraines are unilateral, affecting only half of the head, but in other cases can be bilateral and include neck pain. Although the underlying causes are unknown, genetics and environmental stimulation appear to be significant factors in susceptibility to migraine headaches. Medical research suggests that migraines are a nerurovascular disorder, with symptoms starting in the brain and spreading to blood vessels. Physiological observations and mechanism theories include increased excitability of the cerebral cortex, abnormal control of pain neurons in the trigeminal nucleus of the brain stem, and increased levels of the neurotransmitter serotonin.

Some people do not experience sufficient symptom relief when treated with medications such as valproate, topiramate, or propranolol, but may experience reduce symptoms or occurrence of migraine headaches with biofeedback or neurostimulation. Biofeedback can help people become conscious of and control physiological parameters that may contribute to migraine headaches, such that conscious relaxation of the physiological parameter can reduce incidence or severity of symptoms. Neurostimulation typically involves implanting a device similar to a pacemaker to stimulate the nervous system to treat chronic migraines.

But, using an implantable neurostimulation device is expensive and inconvenient, and reduces the ability of the user to interact with or control the device such as to benefit from biofeedback or other training principles.

Some examples described herein therefore employ an external neurostimulation device worn in or coupled to the ear to treat nervous system disorders such as migraine headaches. In a more detailed example, this is achieved by stimulating at least two nerves at or near their periphery at or near the ear, using an earpiece constructed to fit on or in the ear. The at least two nerves in a further example include a cranial nerve and a spinal nerve.

Stimulation of at least a cranial nerve and a spinal nerve provides for both mediation of cranial pain as experienced with a migraine headache, and disruption of the pain process by providing competing stimulus to disrupt the neural mechanisms normally involved with sensing and mediating pain. The brain discriminates the source of pain poorly, particularly when intense, and having additional sensory input can disrupt the brain's sensation of pain.

In a further example, the user may selectively wear or use the neurostimulation device, such as to train the brain to suppress activity that might otherwise lead to a migraine headache or other condition. Studies have shown that nerve stimulation may modulate or suppress pain signals in the brain, and can modulate or suppress other brain signals and functions. For example, high-frequency nerve excitation using a transcutaneous electrical nerve stimulation (TENS) devices have been shown to decrease pain-related cortical activations in patients with carpal tunnel syndrome, and low-frequency TENS stimulation has been shown to decrease shoulder impingement pain and modulated pain-induced activation in the brain. Similarly, a device marketed as Cefaly has been shown to be effective in preventing migraine attacks, but requires wearing a device on the forehead using adhesive electrodes.

Cranial pain is largely mediated by the 5^{th} cranial nerve, which integrates pain through the spinal nucleus. That nucleus also mediates pain through cranial nerves 7, 9, and 10, and through spinal (cervical) nerves C2 and C3, which may also contribute to the sensation of migraine pain. These nerves converge near the ear, and can be largely stimulated by a single neurostimulation device configured as an earpiece worn on or in the ear. Because nerves are coupled to both the brain and a functional location on the body, electrical stimulation described here is applied at the periphery of the nerve, or at the nerve's functional location on the body, to avoid sending stimulating signals to both the brain and the peripheral functional location on the body. In other examples, stimulation is applied at a point along the nerve where the nerve is accessible to an external neurostimulation device, such as where a nerve not terminating in the ear is accessible as it passes near the ear.

Because neurostimulation is provided by an externally-worn earpiece, the user may easily attach and remove the earpiece, applying stimulation only when needed. Further, the external neurostimulation device can be controlled much more easily than an implantable device, enabling the user to control the neurostimulation device to provide varying degrees of stimulation.

Neurostimulation as described in these examples can be used in various embodiments to treat many types of headaches, and to treat pain in the neck, face, or other parts of the head. Treatment may also be effective for Parkinson's disease, epilepsy, or other neurological disorders, or to reduce symptoms of these or other disorders.

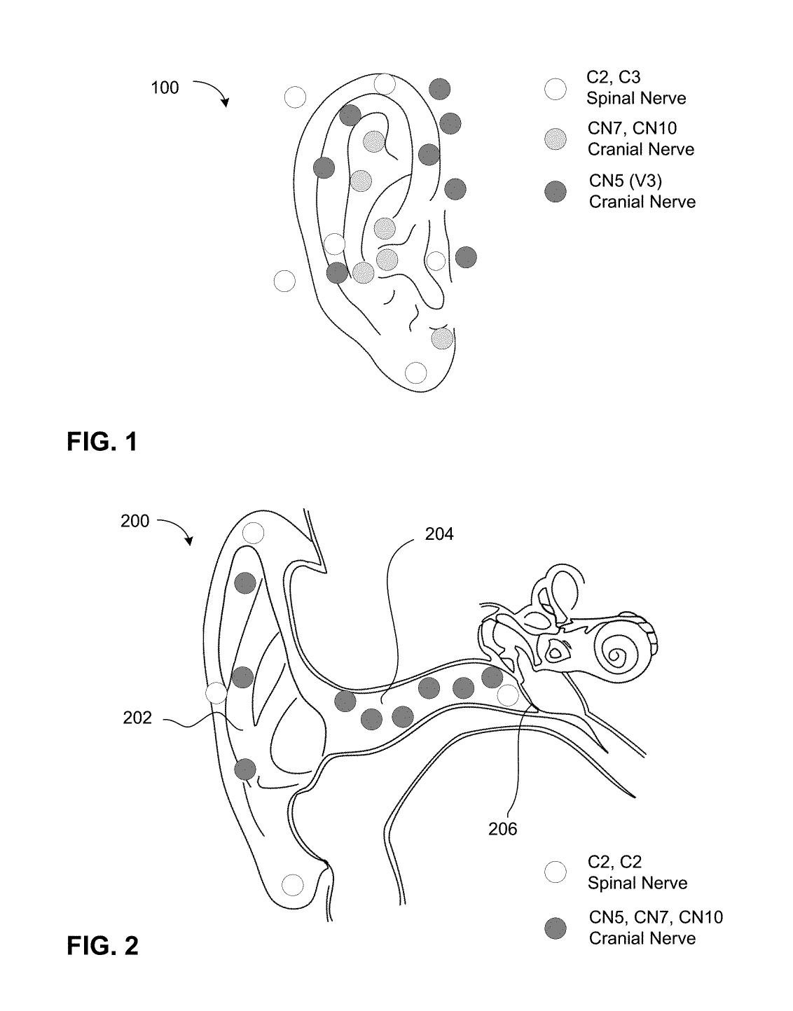

FIG. 1 shows an external view of an ear generally at 100, along with shaded circles indicating nerve access points on and around the ear. Note that the illustration shows both a variety of example spinal and cranial nerve access points, at which an electrical signal could be applied to stimulate the nervous system to relieve pain or treat another nervous system disorder.

FIG. 2 similarly shows a side view of an ear at 200, including both external portions 202 and the external ear canal 204 comprising the portion of the ear canal before the eardrum 206. FIG. 2 also includes shaded circles indicating example access points for various cranial and spinal nerves, with the nerve corresponding to the shaded circle indicated by the degree of shading. As discussed in previous examples, simultaneous or contemporaneous stimulation of the cranial and spinal nerves is desirable in that it may be more effective in alleviating some conditions and symptoms, such as migraine headaches.

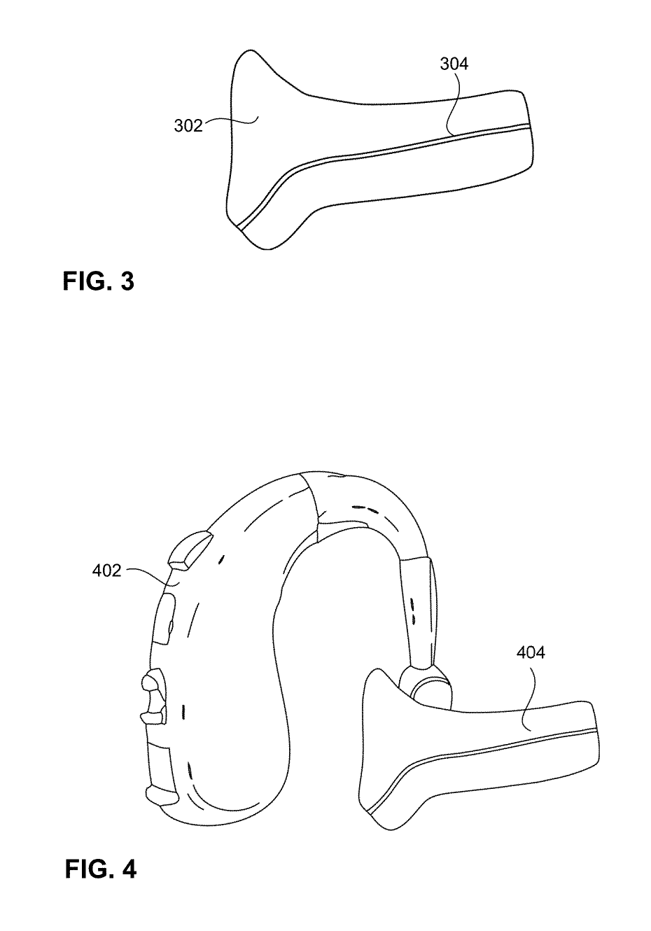

FIG. 3 shows an example insertable electrode earplug 302 that can be inserted into ear canal 206 of FIG. 2, to provide access to nerve points in the ear canal and on the external ear. The electrode earplug 302 in this example includes a hole or hollowed-out portion 304 that enables a wearer to hear while wearing the earplug, and also comprises a number of electrodes corresponding to the access points for various nerves shown in FIGS. 1 and 2. In a further example, the earplug includes one or more grounding or signal return electrodes to provide a complete signal path for the energy supplied to nerves through various electrodes.

FIG. 4 shows an alternate embodiment of an earpiece having electrodes to stimulate nerves in and around the ear, including an external portion 402 that may provide power, user controls, external electrodes, grounding or signal return, and other functions that may not be practically integrated into the insertable electrode earplug 404.

The signals provided through the earpieces or other electrodes comprise in various embodiments alternating current, direct current, steady-state signals, and alternating signals. The type of signal provided and the signal frequency will further vary in some examples depending on the condition being treated, and the intensity of the signal may vary based on the condition or on user input. It is anticipated that signal currents from the microamp to milliamp range may be used to treat such varying conditions, with a user configuring the signal level in a further example by turning the signal level up until a tingling effect is felt and then slightly reducing the provided current. In another example, the signal applied is somewhat randomized, as a randomized signal may be more effective in disrupting the normal cycles involved with pain, migraine headaches, or other such conditions.

The insertable electrode earplug is formed in some examples of a soft material such as silicone, and is a harder material such as molded plastic on other examples. The electrodes are positioned on the earplug and other portions of the earpiece in various examples by using conductive carbon rubber elements, metallic conductors, conductive bumps or pressure points on the exterior of the earpiece, or other suitable methods. In a further example, an electrolyte or other material is used external to the earpiece to enhance conductivity between the earpiece electrodes and the corresponding nerves.

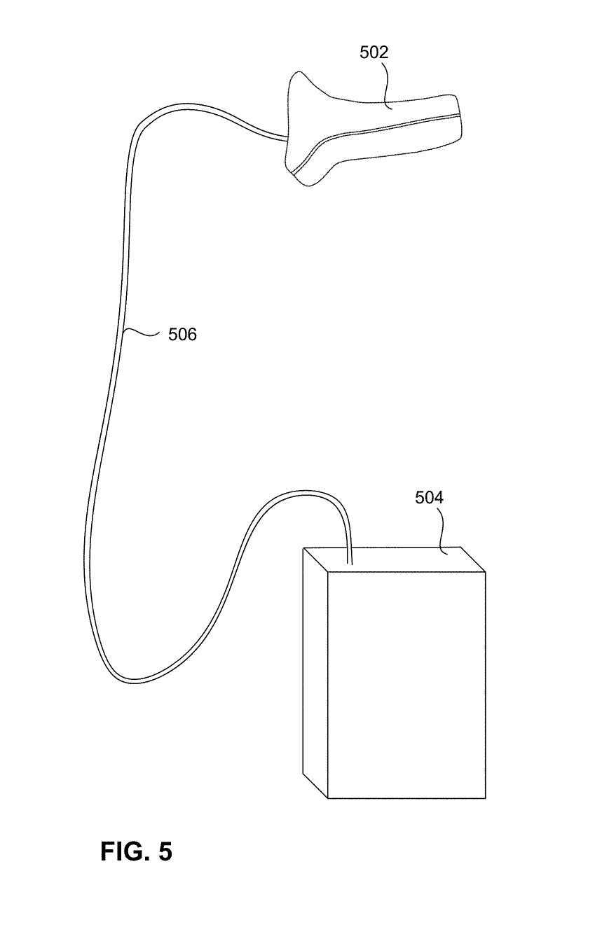

FIG. 5 shows an alternate embodiment in which the earpiece 502 is coupled to a controller 504 via an electrical cable 506. In this example, the earpiece 502 is inserted in, on, or around the ear, while the control module 504 provides external control and power for the electrical ear nerve stimulation system.

Although specific embodiments have been illustrated and described herein, any arrangement that achieve the same purpose, structure, or function may be substituted for the specific embodiments shown. This application is intended to cover any adaptations or variations of the example embodiments of the invention described herein. These and other embodiments are within the scope of the following claims and their equivalents.

1. An electrical ear nerve stimulation earpiece apparatus, comprising:
an earpiece body configured to fit on or in the ear;
a first electrode coupled to a surface of the earpiece configured to stimulate a cranial nerve;
a second electrode coupled to a surface of the earpiece configured to stimulate a spinal nerve
circuitry and operable to provide an electrical signal to at least the first and second electrodes to treat a disorder affecting the nervous system by stimulating the spinal nerve and the cranial nerve.

2. The electrical ear nerve stimulation earpiece apparatus of claim 1, further comprising a power source operable to power the circuitry

3. The electrical ear nerve stimulation earpiece apparatus of claim 2, wherein the power source is contained in the earpiece.

4. The electrical ear nerve stimulation earpiece apparatus of claim 2, wherein the power source is external to the earpiece, and further comprising a power cable coupling the power source to the earpiece.

5. The electrical ear nerve stimulation earpiece apparatus of claim 1, wherein treating the disorder comprises modulating or suppressing signals in a user's brain that contribute to migraine headaches.

6. The electrical ear nerve stimulation earpiece apparatus of claim 1, wherein treating the disorder comprises stimulating cranial nerve 5.

7. The electrical ear nerve stimulation earpiece apparatus of claim 6, wherein treating the disorder further comprises stimulating one or more of cranial nerves 7, 9, and 10.

8. The electrical ear nerve stimulation earpiece apparatus of claim 6, wherein treating the disorder further comprises stimulating at least one of cervical nerves C2 and C3.

9. The electrical ear nerve stimulation earpiece apparatus of claim 1, the disorder comprises migraine headaches, Parkinson's disease, or epilepsy.

10. The electrical ear nerve stimulation earpiece apparatus of claim 1, further comprising an acoustic passage configured to allow a user to hear while wearing the earpiece.

11. A method of stimulating an ear, comprising:
attaching an earpiece configured to fit on or in the ear to a user's ear, the earpiece comprising a first electrode coupled to a surface of the earpiece configured to stimulate a cranial nerve, and a second electrode coupled to a surface of the earpiece configured to stimulate a spinal nerve; and
providing an electrical signal to at least the first and second electrodes to treat a disorder affecting the nervous system by stimulating the spinal nerve and the cranial nerve.

12. The method of stimulating an ear of claim 11, wherein treating the disorder comprises modulating or suppressing signals in a user's brain that contribute to migraine headaches.

13. The method of stimulating an ear of claim 11, wherein treating the disorder comprises stimulating cranial nerve 5.

14. The method of stimulating an ear of claim 13, wherein treating the disorder further comprises stimulating one or more of cranial nerves 7, 9, and 10.

15. The method of stimulating an ear of claim 13, wherein treating the disorder further comprises stimulating at least one of cervical nerves C2 and C3.

16. The method of stimulating an ear of claim 11, the disorder comprises migraine headaches, Parkinson's disease, or epilepsy.

17. The method of stimulating an ear of claim 11, further comprising providing an acoustic passage in the earpiece to allow a user to hear while wearing the earpiece.

18. The method of stimulating an ear of claim 11, further comprising adjusting the electrical signal to provide a current that does not produce a tingling effect to the user.

19. The method of stimulating an ear of claim 11, further comprising randomizing the electrical signal to more effectively disrupt or mask signals related to the disorder being treated.

20. The method of stimulating an ear of claim 11, further comprising providing a ground contact touching the user, such that the electrical signals have a return path through the ground electrode.
